(19) United States
(12) Patent Application Publication
(10) Pub. No.: US 2007/0027363 A1
(43) Pub. Date: Feb. 1, 2007

(54) NERVE PROTECTION SYSTEM

(76) Inventors: James Richard Gannoe, West Milford, NJ (US); John Joseph Flynn, West Milford, NJ (US)

Correspondence Address: GREENBERG TRAURIG, LLP, MET LIFE BUILDING, 200 PARK AVENUE, NEW YORK, NY, 10166, US

(21) Appl. No.: 11/460,734

(22) Filed: Jul. 28, 2006

Related U.S. Application Data
(60) Provisional application No. 60/703,546, filed on Jul. 28, 2005.

(51) Int. Cl. A61B 1/32

(52) U.S. Cl. 600/215; 600/206

(57) ABSTRACT

The present invention relates to a device (and corresponding method) that protects (e.g., shields and/or retracts) exiting and/or descending nerve (e.g., nerve root). This may be carried out in a controlled, unobtrusive manner during, for example, Transforaminal Lumbar Interbody Fusion (“TLIF”), Posterior Lumbar Interbody Fusion (“PLIF”), or Minimally Invasive Surgery (“MIS”) spinal procedures. Further, the device may be form-fit to one or more particular ones of the following: nerve, pedicle, annulus, vertebral body, interbody device.

### RELATED APPLICATIONS

This application claims the benefit of U.S. Provisional Application Ser. No. 60/703,546, filed Jul. 28, 2005, which is incorporated herein by reference in its entirety.

### FIELD OF THE INVENTION

The present invention relates to a device (and corresponding method) that protects (e.g., shields and/or retracts) exiting and/or descending nerve (e.g., nerve root). This may be carried out in a controlled, unobtrusive manner during, for example, Transforaminal Lumbar Interbody Fusion (“TLIF”), Posterior Lumbar Interbody Fusion (“PLIF”), or Minimally Invasive Surgery (“MIS”) spinal procedures.

Further, the device may be form-fit to one or more particular ones of the following: nerve, pedicle, annulus, vertebral body, interbody device.

### BACKGROUND OF THE INVENTION

In TLIF procedures using a Harms (or other) technique, the nerve roots adjacent to the spinal column are exposed by the surgical approach. The surgeon will create a channel accessing the disc space via facetotomy/facetectomy (TLIF). By removing the bony and cartilaginous anatomy via these maneuvers, the surgeon exposes the exiting nerve roots at the operative level. These nerve roots are vulnerable to damage via blunt trauma, laceration, scraping, crushing, etc. during passage of the various instruments to perform discectomy, disc space preparation, and interbody placement. A surgeon may need to retract the nerve root medially, laterally, caudally or cephalad depending on the patient, to gain adequate access and visualization to the disc space. It is well known that tensioning or traumatizing a nerve root can lead to post operative radiculopathy, numbness, and ataxia, among other things. It is also understood that an increase in the exposure and manipulation of the nerve root can lead to increased nerve root fibrosis, inflammation and other side effects postoperatively. As spinal fusion procedures continue to be developed, the need exists to perform more significant preparative and restorative surgery within the disc space. This need is driving the use of more aggressive techniques and the use of more sophisticated implants. Also, as this technique becomes more popular, it is being attempted by less skilled surgeons. The risk of nerve injury in general is increasing.

There is a trend in spinal surgery toward tissue sparing techniques. These techniques employ tubular and expanding retraction devices that preserve the musculature and soft tissue structures surrounding the spine. As a result, the access and visualization provided to the surgeon is diminishing. As the incisions become smaller, the need for removing instrument “clutter” is increasing. The need for devices that address this issue is growing.

One embodiment of the present invention helps to mitigate many of the risks of posterior and posterio-lateral interbody fusion by providing a nerve protecting element. Another embodiment of the present invention facilitates these procedures by helping to reduce the level of skill and dexterity required to avoid the nerve roots with instruments and implants. Another embodiment of the present invention provides nerve root protection without unduly cluttering the operative site with handles and instrument shafts.

### BRIEF DESCRIPTION OF THE DRAWINGS

FIG. 1 shows a view of the Lumbar anatomy (more particularly, a posterior view of the Lumbar spine showing exposed nerve roots, Cauda Equina and Dura);

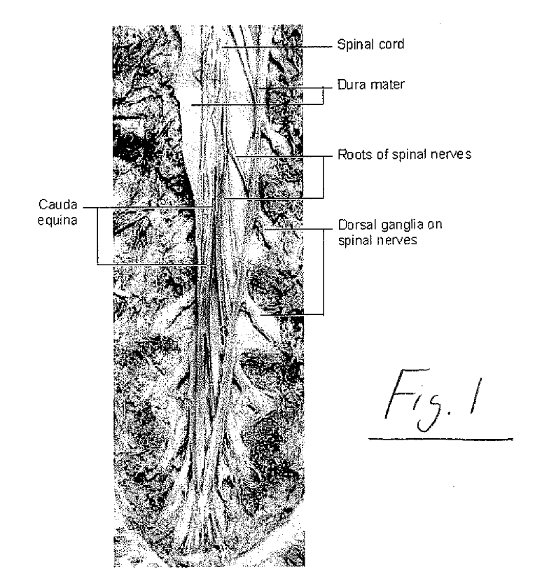

FIG. 2 shows a spring loaded/specula style deployable spring clip according to an embodiment of the present invention;

FIGS. 3A and 3B show an expanding sleeve/stent according to an embodiment of the present invention;

FIG. 4 shows a Penfield 4/Retractor blade according to an embodiment of the present invention;

FIG. 5 shows a PLIF approach dura/traversing nerve protector according to an embodiment of the present invention; and

FIG. 6 shows a TLIF approach exiting nerve protector according to an embodiment of the present invention.

Among those benefits and improvements that have been disclosed, other objects and advantages of this invention will become apparent from the following description taken in conjunction with the accompanying figures. The figures constitute a part of this specification and include illustrative embodiments of the present invention and illustrate various objects and features thereof.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

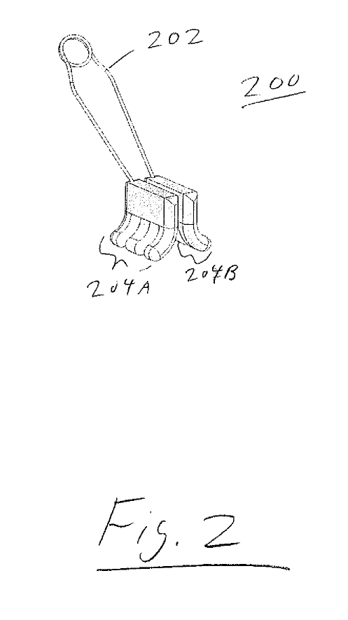

Referring now to FIG. 2, a spring loaded/specula style deployable spring clip embodiment is shown. As seen in this Fig., this embodiment may comprise a generally U-Shaped Device 200 formed of Spring Element 202 having a number of Blades 204A and 204B on the ends thereof (there are three blades on each end of Spring Element 202 in this example, although only one of the blades is seen in this view at the position of Blades 204B). The U-Shaped Device 200 may be spring loaded via Spring Element 202 to create a distraction type force when positioned in the body (e.g., between the nerve and opposite pedicle). To use, the device may be compressed, Blades 204A and 204B inserted between nerve and opposite pedicle (or other fixed point), released, and allowed to distract and protect the nerve. The device may stay in place during the entire procedure, or may be periodically removed if intermittent retraction is necessary (if periodically removed, the device may be disposed of after each removal and replaced or may be re-used). Of course, FIG. 2 is provided as an example (which example is intended to be illustrative and not restrictive), and the device may take any desired form (e.g., the blade(s) may be of varying numbers, shapes and materials to provide optimal shielding and retraction characteristics for a particular use).

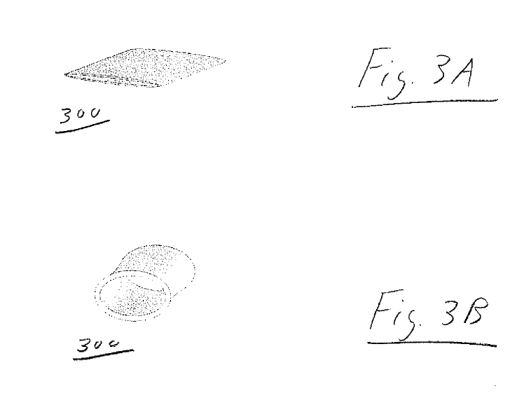

Referring now to FIGS. 3A and 3B, an expanding sleeve/stent embodiment is shown. As seen in these Figs., this embodiment may comprise a Stent-Style Device 300 that is initially implanted in a collapsed position (see FIG. 3A), placed in an appropriate location, and allowed to expand (see FIG. 3B) by internal spring/features, material properties, and/or through use of an instrument. Once expanded (e.g., in vivo to provide a working channel) the shape and position of the device provides protection (e.g., for exiting nerve). In one example (which example is intended to be illustrative and not restrictive), the device may have spikes or barbs (e.g., on a distal end and/or a length of the device) that puncture or otherwise engage an outer portion of an annulus or vertebral body to secure in place.

Referring now to a caliper style embodiment of the present invention, it is noted that this embodiment may comprise a device that has dual parallel blades that are spring loaded with internal cable (similar to a bike caliper system, with opposite force delivery). This device may be implanted in a collapsed condition, opened (e.g., using a distractor type instrument), and a set screw secured that fixes the device in the open position (in another example (which example is intended to be illustrative and not restrictive), compression spring(s) may maintain the open position of device). Of note, if desired, the cable may be released periodically to allow for intermittent distraction.

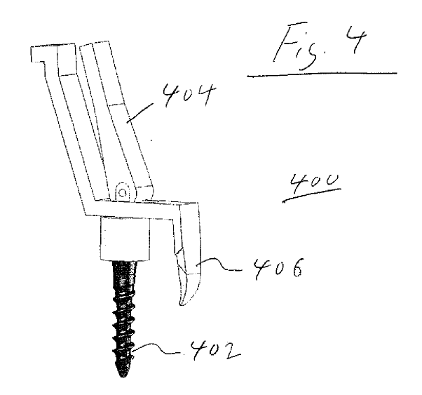

Referring now to FIG. 4, a Penfield 4/Retractor blade embodiment is shown. As seen in this Fig., this embodiment may comprise Device 400 adapted to be attached to a fixed point within the patient's body. More particularly, Screw 402 (e.g., a pedicle screw) may be attached to a pedicle of the patient (in one example (which example is intended to be illustrative and not restrictive), the pedicle screw may be attached to the patient in conjunction with another device or system, such as the ICON system from BLACKSTONE MEDICAL). Further, Locking Member 404 (which may, for example, removably snap on to the head of Screw 402) may serve to releasably lock Blade 406 in place as desired (the locking may be carried out, for example, by friction and cam-action). Further still, the Device 400 device may include a feature to allow for manipulation of Blade 406 to engage and retract nerve root (in one example (which example is intended to be illustrative and not restrictive), Blade 406 can slide and/or pivot to provide retraction). Once the nerve root is retracted, Blade 406 can be locked in place using Locking Member 404 to maintain retraction. Further, if desired, Locking Member 404 can be released periodically to allow intermittent retraction. Of course, FIG. 4 is provided as an example (which example is intended to be illustrative and not restrictive), and the device may take any desired form (e.g., the blade(s) may be of varying numbers, shapes and materials to provide optimum shielding and retraction characteristics for a particular use).

As mentioned above, the pedicle screw may be attached to the patient in conjunction with another device or system, such as the ICON system from BLACKSTONE MEDICAL. In another example, the nerve protector may be used in connection with an SFS screw head and/or MIS retraction system).

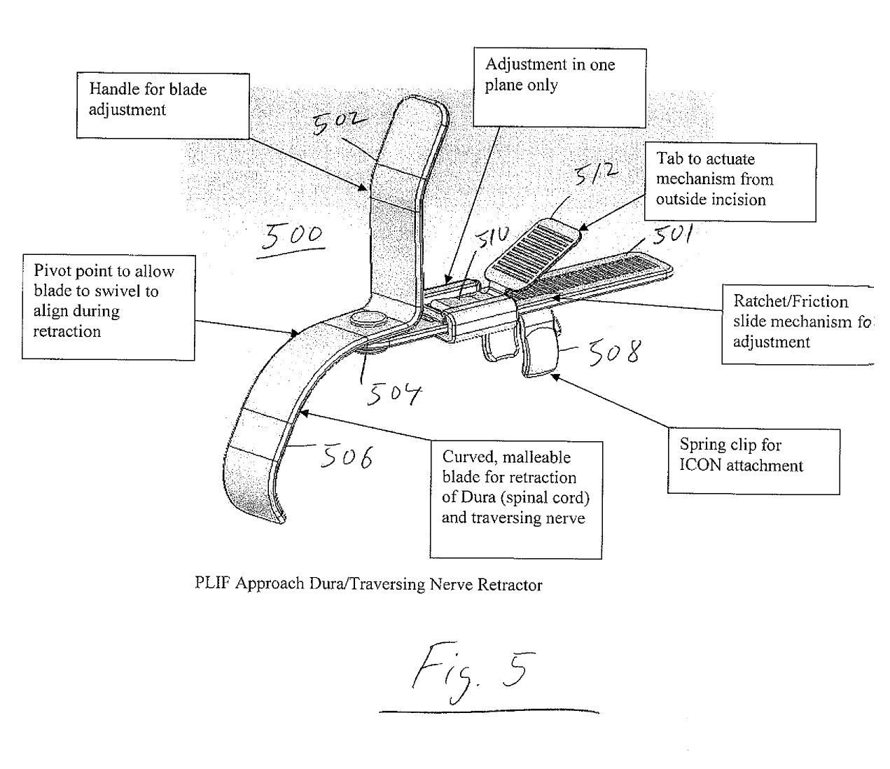

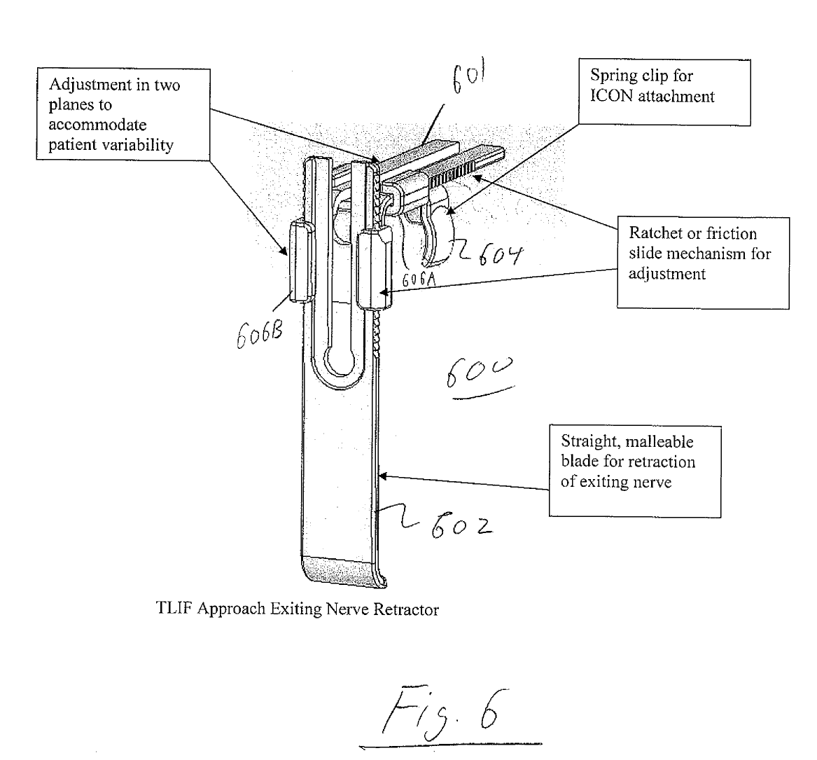

Referring now to FIGS. 5 and 6, additional embodiments of the present invention are shown. In these two Figs., two distinct versions of a device configured to be utilized for the TLIF and PLIF approaches, respectively, are shown. Of note, differences between the TLIF and PLIF devices of these FIGS. 5 and 6 are the length and general shape of the blade that performs the retraction, the type and direction of adjustment and the anatomical elements that are retracted.

More particularly, it is seen that the PLIF device (see FIG. 5) attaches to the pedicle screw on the contra-lateral (opposite) side of which the implant will be inserted, and the dura (spinal cord) and traversing nerve root retracted.

In contrast, the TLIF device (see FIG. 6) attaches to the pedicle screw placed in the superior pedicle on the operative side, allowing retraction of the exiting nerve root at the operative level.

Referring now more particularly to FIG. 5, it is seen that Device 500 includes Body 501 and Handle 502 (e.g., for blade adjustment). Further, Pivot Point 504 may be provided to allow Blade 506 to swivel relative to Body 501 to align during retraction. Further still, Blade 506 may be curved and/or malleable (e.g., for retraction of Dura (spinal cord) and traversing nerve). Further still, Spring Clip 508 may be provided for attachment to an ICON device (or any other desired device placed within the patient). Further still, Ratchet and/or Friction Slide Mechanism 510 may be provided for adjustment (e.g., sliding of Body 501 relative to Spring Clip 508). Further still, Tab 512 may be provided (e.g., to actuate Slide Mechanism 510 from outside the incision). Of note, as seen in this FIG. 5, the slide adjustment may be (in this example) along one plane).

Referring now more particularly to FIG. 6, it is seen that Device 600 includes Body 601 and Blade 602. Blade 602 may be straight and/or malleable (e.g., for retraction of exiting nerve). Further, Spring Clip 604 may be provided for attachment to an ICON device (or any other desired device placed within the patient). Further still, Ratchet and/or Friction Slide Mechanism 606A and 606B may be provided for adjustment (e.g., sliding Body 601 relative to Spring Clip 604 (in the case of Slide Mechanism 606A) and sliding Blade 602 relative to body 601 (in the case of Slide Mechanism 606B)). Of note, as seen in this FIG. 6, adjustment may be (in this example) in two planes to accommodate patient variability.

As described above, various embodiments of the present invention may utilize a snap-on feature that allows for attaching the nerve protection device to the head of the screw (e.g., the ICON screw). In another embodiment, the nerve protection device may be attached to the screw by means other than (or in addition to) a snap-on connection. In another embodiment, an adjustment mechanism may incorporate a friction slide mechanism that allows for control of the adjustment along essentially an infinite number of positions within a range. In another embodiment, a ratchet mechanism may be utilized for blade control/movement.

In another embodiment, the shape of the blade(s) may accommodate the direction of the retraction relative to the anatomy. In another embodiment, adjustment of the blade length (e.g., in the anterior/posterior direction for the TLIF version) may be utilized to accommodate variability in patient anatomy.

Reference will now be made to an example use of the present invention for improving the safety and ease of a transforaminal lumbar interbody fusion (TLIF) procedure performed posteriorly. The device and method of this example allow a physician to protect (e.g., shield and if needed, retract) the exiting and traversing nerve roots during this spinal surgery. The invention contemplates disposable and reusable embodiments of a device that is positioned in the surgically created access channel post facetotomy/facetectomy or other access maneuver.

In TLIF procedures using a Harms (or other) technique, the nerve roots adjacent to the spinal column are exposed by the surgical approach. The surgeon will create a channel accessing the disc space via facetotomy/facetectomy (TLIF). By removing the bony and cartilaginous anatomy via these maneuvers, the surgeon exposes the exiting nerve roots at the operative level. These nerve roots are vulnerable to damage via blunt trauma, laceration, scraping, crushing, etc. during passage of the various instruments to perform discectomy, disc space preparation, and interbody placement. A surgeon may need to retract the nerve root medially, laterally, caudally or cephalad depending on the patient, to gain adequate access and visualization to the disc space. It is well known that tensioning or traumatizing a nerve root can lead to post operative radiculopathy, numbness, and ataxia, among other things. It is also understood that an increase in the exposure and manipulation of the nerve root can lead to increased nerve root fibrosis, inflammation and other side effects postoperatively. As spinal fusion procedures continue to be developed, the need exists to perform more significant preparative and restorative surgery within the disc space. This need is driving the use of more aggressive techniques and the use of more sophisticated implants. Also, as this technique becomes more popular, it is being attempted by less skilled surgeons. The risk of nerve injury in general is increasing.

There is a trend in spinal surgery toward tissue sparing techniques. These techniques employ tubular and expanding retraction devices that preserve the musculature and soft tissue structures surrounding the spine. As a result, the access and visualization provided to the surgeon is diminishing. As the incisions become smaller, the need for removing instrument “clutter” is increasing. The need for devices that address this is growing.

One embodiment of the present invention helps to mitigate many of the risks of posterior and posterio-lateral interbody fusion by providing a nerve protecting device. Another embodiment of the present invention facilitates these procedures by helping to reduce the level of skill and dexterity required to avoid the nerve roots with instruments and implants. Another embodiment of the present invention provides nerve root protection (e.g., retraction) without unduly cluttering the operative site with handles and instrument shafts.

In the present example, the device is a deployable, disposable protector that can be inserted into the facetotomy/facetectomy. The device has at least one and conceivably a plurality of root protecting elements, such as one or more blades or barriers that extends anteriorly from the posterior aspect of the spine and serves to protect the nerve root(s) from incidental and accidental contact with any devices. Such contact typically occurs during annulotomy, discectomy, disc space and endplate preparation and interbody implant and graft placement. Instruments used in these maneuvers are typically of rigid construction and have edges, corners and protrusions that can damage nerve tissues. The present example also has an expansion capability that provides retention and retraction forces when the device is deployed. For example, the device may be spring loaded to achieve an expanded condition once released by a deployer. The blades or barriers may be designed to conform closely to the anatomy so as to provide the optimal access channel into the disc space. This expanded condition would allow a retention and retraction force to develop between the device and anatomy. The present example would be low profile, unobtrusive and would be designed not to impede the surgeon's motions, access and visualization of the operative site. A removable handle or deployer is contemplated that allows the surgeon to position and/or mount the device and subsequently detach and remove the handle prior to interbody surgery. The detachable handle would then be re-applied to remove and/or reposition the device.

Of course, the above description of this example has been applied to posterior spinal fusion. However, it should be apparent to those skilled in the art that the technology described could be applied to any surgery in which nerve protection in a similar manner would benefit the patient and/or the physician.

Referring now to Table 1, below, various examples for a number of parameters associated with a device according to an embodiment of the present invention are shown (of course, the examples of this Table 1 are intended to be illustrative and not restrictive).

TABLE 1

| | |
|---|---|
| Parameter | Examples |

Assembly Specifications

| | |
|---|---|
| Distraction Sizes | Single, multiple |
| Device stability | Mounted, Unmounted |
| Open position | Locking, unlocking |
| Distraction force delivery | Continuous, intermittent |
| Durability | Reusable, disposable |
| Sterility | Sterile, non-sterile |
| Actuation | Spring loaded, manual, |
| | cables |
| Retraction amount | 0-10 mm |
| Retraction increments | 1 mm, 2 mm |
| Working height | 6 mm-16 mm |
| Procedures | TLIF, PLIF, |
| | Discectomy |

Protection Element

| | |
|---|---|
| Material | Stainless Steel, Nitinol, |
| | PEEK, Polycarbonate, |
| | Silicone, Polyolefin, |
| | Polyester, Polyurethane, |
| | Nylon, Teflon, Kevlar |
| Height | .25″-1″ |
| Width | .05″-.5″ |
| Thickness | .010″-.050″ |
| Anterior/Posterior (Front to | Concave to fit pedicle |
| back) Profile | flat, |
| Axial (top down) Profile | Form fit to vertebral |
| | body, annulus |
| Lateral Profile | Form fit to nerve |
| Collapsed profile | Bullet nosed, wedge |
| | shape, flat |
| Color | Clear, matte, opaque, |
| | radio-lucent, radio- |
| | opaque |
| Finish | Smooth, mirror, matte, |
| | non-tissue binding |
| Type | Spring, Cable |

Actuation element

| | |
|---|---|
| Material | SST, Nitinol, Other |
| Shape | Torsion spring, |
| | “U”shaped spring, |
| | Compression spring |
| Length | .1-4″ |
| Diameter | .005″-.050″ |
| K Factor | Low force |

In various embodiments the device may be reusable. It is also contemplated that the device may be mounted, either to the anatomy, a retractor (such as those used in tissue sparing techniques) a cannula and/or the table. The device may “clip on” to the anatomy or a retractor. It is also contemplated that the device may be mounted to a previously implanted pedicle screw or other fixation device. In one example (which example is intended to be illustrative and not restrictive), the device may attach to the screw post of a modular pedicle screw.

In another embodiment, the device may not expand. Rather, the device may be designed such that the monolithic or non-actuating form factor achieves the desired balance of ease of placement/use and access/visualization.

Material characteristics which may be used in the design of the device include (but are not limited to) radiolucency, transparency, biocompatibility, manufacturability, mechanical barrier/shielding properties, and pyrogenicity. The materials used in the construction may include (but not be limited to) stainless steel, nitinol, polycarbonate, polyolefin, polyester, polyurethane, silicone, nylon, PEEK and/or other similar material (or any combination thereof).

Of note, it is the preference of some surgeons to intermittently retract and relax the nerve root in procedures that require root retraction. Thus, in an embodiment of the present invention, the access channel created by the device could be dynamic. The physician could control whether the root was retracted or relaxed (and the extent thereof) via an actuation cable or equivalent. It is also contemplated that the device could be “urged” open by the tip of an instrument as it was inserted. As the instrument was removed, the device could re-close. The result would be intermittent retraction.

In yet another embodiment, the nerve contacting end effectors may be narrow and may be similar in geometry to nerve root retractors used in traditional surgery such as a Penfield #1 or #4.

In yet another embodiment, the device could have interchangeable end effectors to deal with varying anatomy and/or varied surgeon preferences.

Of note, various embodiments of the present invention provide for one or more of the following: (a) ability to repeatedly enter disc space with low risk of nerve damage; (b) improved force delivery during shielding and retraction by applying controlled, repeatable, reliable forces to nerve; (c) eliminates (or reduces) need for assistance from attending surgeon; (d) reduction in incision “clutter”.

While a number of embodiments of the present invention have been described, it is understood that these embodiments are illustrative only, and not restrictive, and that many modifications may become apparent to those of ordinary skill in the art. For example, any steps described herein may be carried out in any desired order (and any additional steps may be added as desired and any steps may be deleted as desired).

What is claimed is:

1. A nerve protection apparatus, comprising:
an attaching mechanism, wherein the attaching mechanism attaches to a head of a pedicle screw;
a body having a first end and a second end, wherein the body is connected to the attaching mechanism, and wherein the body is connected to the attaching mechanism such that the body may be slid relative to the attaching mechanism;
a generally curved blade piviotally mounted to the body adjacent the first end of the body;
a handle connected to the blade, wherein the handle pivots the blade relative to the body; and
a locking mechanism, wherein the locking mechanism releasably locks a position of the body relative to the attaching mechanism.

2. The apparatus of claim 1, wherein the apparatus is configured for use in connection with a PLIF procedure.

3. The apparatus of claim 1, wherein the blade is malleable.

4. The apparatus of claim 1, further comprising a tab, wherein the tab is configured to actuate the locking mechanism.

5. The apparatus of claim 1, wherein the locking mechanism comprises a frictional locking mechanism.

6. The apparatus of claim 1, wherein the locking mechanism comprises a ratchet locking mechanism.

7. The apparatus of claim 1, wherein the attaching mechanism snaps on to the head of the pedicle screw.

8. The apparatus of claim 1, wherein the apparatus is configured for retracting a nerve within a patient.

9. The apparatus of claim 8, wherein the blade is configured to retract a dura/traversing nerve.

10. A nerve protection apparatus, comprising:
an attaching mechanism, wherein the attaching mechanism attaches to a head of a pedicle screw;
a body having a first end and a second end, wherein the body is connected to the attaching mechanism, wherein the body is connected to the attaching mechanism such that the body may be slid relative to the attaching mechanism, and wherein the movement of the body relative to the attaching mechanism is along a first axis;
a generally straight blade, wherein the blade is connected to the body adjacent the first end of the body, and wherein the blade is connected to the body such that the blade may be slid relative to the body along a second axis generally perpendicular to the first axis;
a first locking mechanism, wherein the first locking mechanism releasably locks a position of the body relative to the attaching mechanism; and
a second locking mechanism, wherein the second locking mechanism releasably locks a position of the blade relative to the body.

11. The apparatus of claim 10, wherein the apparatus is configured for use in connection with a TLIF procedure.

12. The apparatus of claim 10, wherein the blade is malleable.

13. The apparatus of claim 10, wherein the first locking mechanism comprises a frictional locking mechanism.

14. The apparatus of claim 10, wherein the first locking mechanism comprises a ratchet locking mechanism.

15. The apparatus of claim 10, wherein the second locking mechanism comprises a frictional locking mechanism.

16. The apparatus of claim 10, wherein the second locking mechanism comprises a ratchet locking mechanism.

17. The apparatus of claim 10, wherein the attaching mechanism snaps on to the head of the pedicle screw.

18. The apparatus of claim 10, wherein the apparatus is configured for retracting a nerve within a patient.

19. The apparatus of claim 18, wherein the blade is configured to retract an exiting nerve.
